(19) United States
(12) Patent Application Publication
(10) Pub. No.: US 2012/0230865 A1
(43) Pub. Date: Sep. 13, 2012

(54) Method and an installation for sterilizing containers by electron bombardment

(75) Inventors: André GRAFFIN, Winfield, IL (US); Guy Dumargue, Cherre (FR); Delphine Gueguen, Cherreau (FR)

(73) Assignee: Serac group, La Ferte Bernard (FR)

(21) Appl. No.: 13/418,171

(22) Filed: Mar. 12, 2012

(30) Foreign Application Priority Data
Mar. 10, 2011 (FR) 1151996

(51) Int. Cl. A61L 2/02; A61L 2/08

(52) U.S. Cl. 422/22; 250/453.11

(57) ABSTRACT

A method of sterilizing containers by means of an electron gun, the method comprising the steps of inserting the gun into an opening of the container and of applying electron bombardment, and also the step of orienting the container so that it has its opening directed downwards prior to inserting the gun therein. The invention also provides an installation for implementing the method.

### FIELD OF THE INVENTION

The present invention relates to a method and to an installation for sterilization by electron bombardment.

By way of example, such an installation is designed to be included in a production line for filling containers, upstream from the filling proper.

### BACKGROUND OF THE INVENTION

Sterilization installations are known that make use of high temperature treatments or of chemical treatments, in particular using hydrogen peroxide.

Those sterilization installations nevertheless impose considerable constraints, in particular in terms of process. For example, using a high temperature leads to the containers becoming heated and it may be necessary to cool them before they are filled in order to avoid spoiling the substance with which they are filled. The use of chemicals may lead to the need for the containers to be rinsed before they are filled. Furthermore, the effectiveness of the treatment depends directly on the duration for which the containers are subjected to the high temperature or are in contact with the treatment chemical. The use of high temperature is also undesirable with certain containers that would run the risk of deforming during sterilization.

A sterilization treatment technique making use of electron bombardment has been available for several years, which technique consists of subjecting the insides of containers to a stream of electrons from an electron gun inserted directly into the openings of the containers. A sterilization installation comprises a platform for transporting containers, which platform carries electron guns, container holder members in register with the electron guns, and relative movement means for moving the guns relative to the containers in order to insert free ends of the guns into the openings of the containers. More precisely, the guns extend above the containers and the movement means serve to raise and lower the containers relative to the guns.

The treatment with electrons is found to be particularly effective, while limiting the constraints that are induced on the process. Nevertheless, the installation is complex and bulky.

### OBJECT AND SUMMARY OF THE INVENTION

An object of the invention is to provide means for resolving the above-mentioned drawbacks, at least in part.

To this end, the invention provides a method of sterilizing containers by means of an electron gun, the method comprising the steps of inserting the gun into an opening of the container and of applying electron bombardment. The method further comprises the step of orienting the container so that its opening is directed downwards prior to inserting the gun therein.

The invention also provides an installation for sterilizing containers, the installation comprising a container transport platform, the platform having electron guns, container holder members in register with the electron guns, and relative movement means for moving the guns and the containers relative to one another in order to insert free ends of the guns into the openings of the containers. The guns are directed upwards and the holder members are arranged above the electron guns.

Thus, the guns occupy the bottom portion of the installation, thereby releasing the top portion of the installation and simplifying the electrical power supply to the guns. Furthermore, in installations that include means for diffusing a laminar stream of air, it is desirable to have as few elements as possible in the top portion of the installation so as to avoid disturbing the air stream. In addition, the upside-down position of the containers makes it easier to remove any impurities that might be held captive inside the containers. When external treatment is also applied to the containers, having their openings directed downwards makes it easier to treat the bottoms of the containers. This arrangement also makes it easier to protect operators from the electrons. Finally, the installation is also easier to clean and sterilize.

In addition, while sterilizing containers, ozone may form in the containers. Having the containers upside-down enables the ozone that is formed to be trapped inside the containers until the containers are taken to a zone of the installation that is provided with ozone extractor devices to ensure that the release of the ozone to the outside of the containers is controlled. Ozone exhaust is thus under control. In order to prevent ozone forming, it is possible to inject nitrogen into the containers. Having the containers upside-down enables nitrogen to be trapped inside the containers without any need to close said containers.

Advantageously, the guns are stationary vertically and the movement means are arranged to vary the height of the holder means relative to the guns.

Moving the containers is subjected to fewer constraints than moving the guns, in particular because the guns are connected to a considerable amount of wiring. In addition, the containers are lighter than the guns, thereby simplifying the structure of the movement means and reducing the weight thereof.

According to a particular characteristic, the installation includes an enclosure with a controlled atmosphere in which the holder members and the free ends of the guns extend and that has a bottom transverse wall through which the guns pass in leaktight manner.

The enclosure can be sealed relative to the outside environment in particularly simple manner.

Preferably, the installation has tilt means for tilting the holder means about the gun.

The tilt means enable a larger fraction of the bottom of the container to be subjected to the electron stream, in particular when the bottom of the container includes a central bulge projecting into the inside of the container.

It is then advantageous for the tilt means to be arranged to cause a longitudinal axis of the container to rock on either side of a central axis of the gun in a plane perpendicular to a travel direction of the containers.

Since the containers are transported side by side, rocking in a plane perpendicular to the travel direction does not lead to any risk of mutual interference between the containers.

### BRIEF DESCRIPTION OF THE DRAWINGS

Other characteristics and advantages of the invention appear on reading the following description of particular, non-limiting embodiments of the invention.

Reference is made to the accompanying drawings, in which:

FIG. 1 is a perspective view of a sterilization installation in accordance with the invention;

FIG. 2 is a perspective view of one station of the installation;

FIG. 3 is a perspective view of a gripper head of the installation, shown in a first extreme orientation of the container holder means, this view showing the gripper heads of the installation in FIGS. 1 and 2 on a larger scale;

FIG. 4 is a view analogous to FIG. 3, showing the container holder means in a second extreme orientation;

FIG. 5 is an axial section view of the gripper head in an intermediate orientation of the container holder means; and

FIG. 6 is a fragmentary half-view in axial section of an installation in accordance with the invention.

### MORE DETAILED DESCRIPTION

The invention relates to a method and an installation for sterilizing containers, which method and installation are described below with reference to the figures.

Each container 100 comprises a body having one end closed by a bottom and an opposite end extended by a neck that defines an opening.

Sterilization is performed by means of electron guns, themselves known, each comprising a generator 1 extended by an emitter 2.

The method comprises the steps of:
orienting the containers so that they have their openings directed downwards; and
inserting the emitter 2 of each gun 1 into an opening of a container while the gun 1 is emitting a stream of electrons.

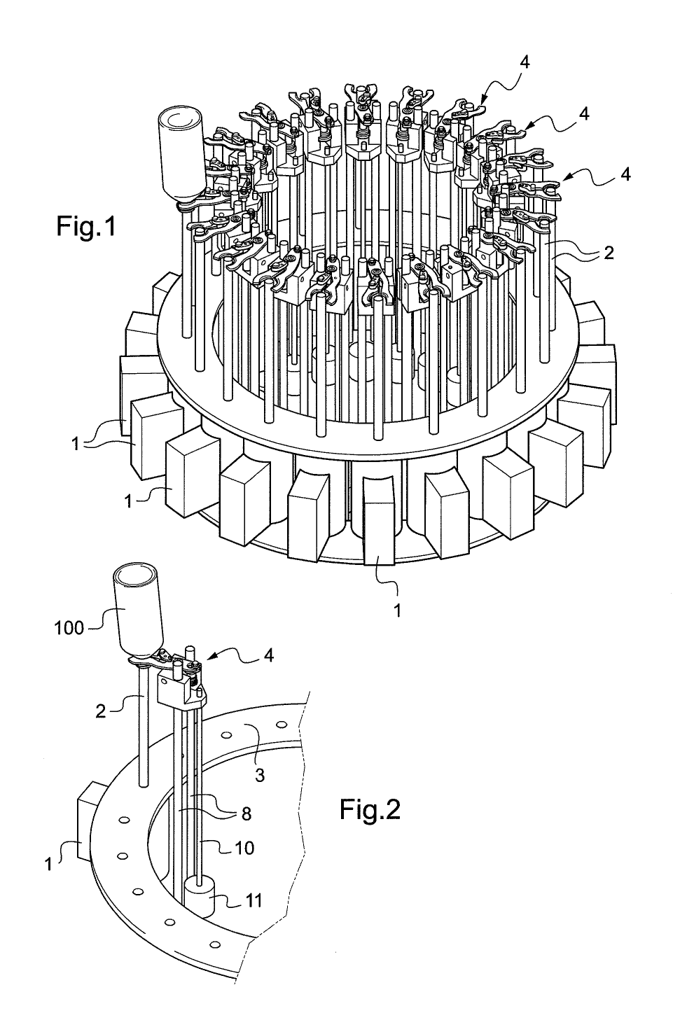

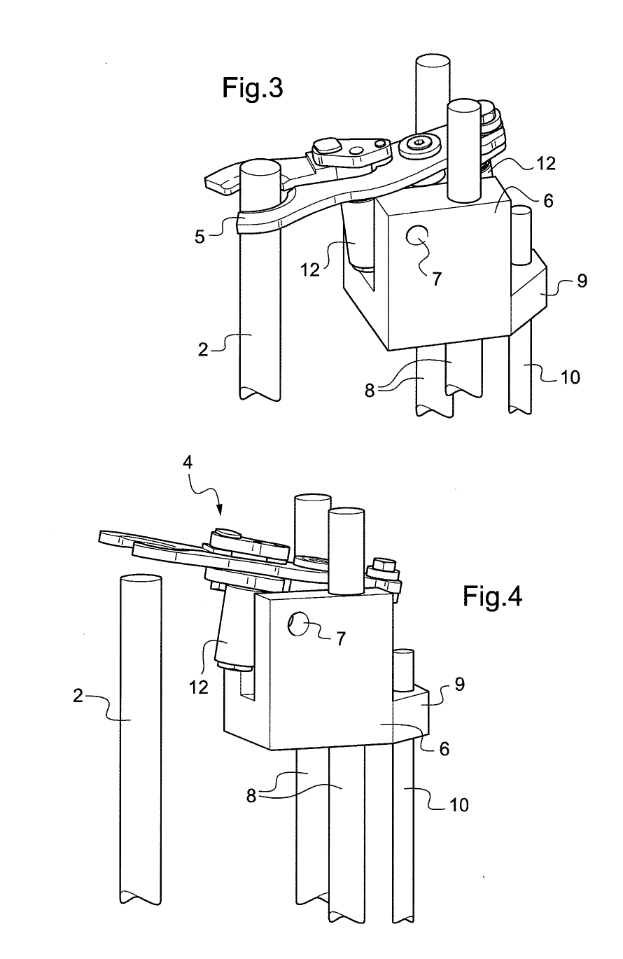

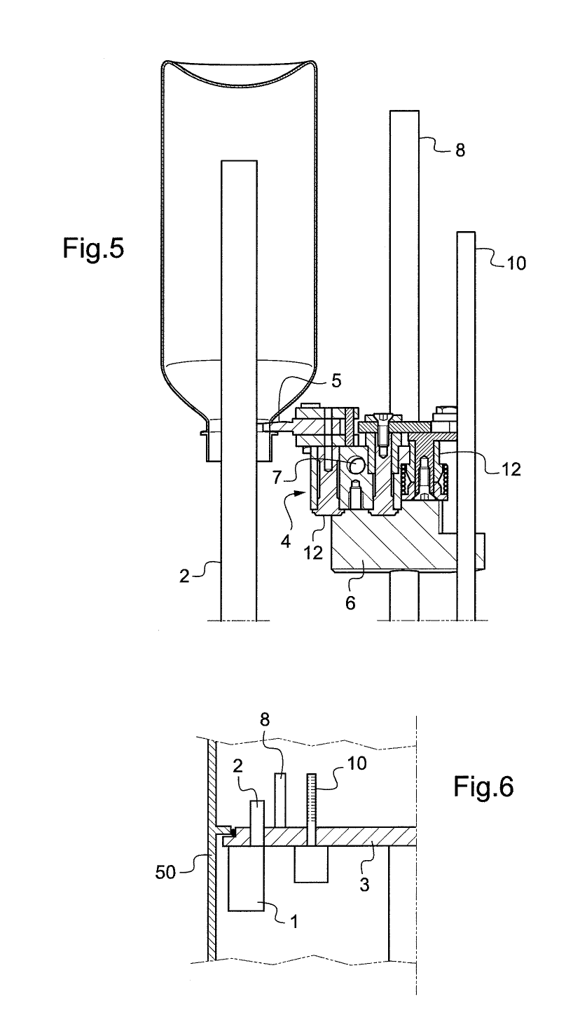

The method is implemented by means of a container sterilization installation having a platform 3 for transporting containers, which platform is mounted to turn on a stand (not shown) and is tangential to a platform for loading and to a platform for unloading, which unloading and unloading platforms are not shown but are themselves known. The platform 3 is in the form of a disk, but only an annular portion thereof is shown in FIGS. 1 to 5. The loading platform receives the containers with their openings directed upwards and in known manner it is arranged to turn the containers upside-down so that they have their openings directed downwards, and to load the containers while in this orientation onto the platform 3. The unloading platform receives the containers with their openings directed downwards and, in known manner, it is arranged to turn them the right way up so that they have their openings directed upwards for subsequent filling.

The platform 3 carries the electron guns, which are mounted on the platform 3 in such a manner that the generators 1 extend beneath the platform 3, while the emitters 2 extend vertically, projecting above the platform 3. The electrical power supply to the generators 1 comprising wiring is mounted in the vicinity of the generators 1 under the platform 3 and is connected to an electrical power source by means of rotary connectors that are themselves known.

Container holder members 4 are mounted on the platform 3 in register with the electron guns, and they are associated with relative movement means for moving the guns and the containers relative to one another in order to introduce free ends of the emitters 2 of the guns into the openings of the containers.

The holder members 4 are arranged above the electron guns, and each of them includes a clamp 5 having jaws arranged to clamp the sides of the neck between a rim and the body of the container, on either side of a midplane of the container that coincides with a radial plane of the platform 3. The clamp 5 may be controlled by means of a cam that is stationary relative to the stand, or by an actuator of the servo-motor or cylinder type.

Since the guns are of constant height in this example, the movement means are arranged to vary the height of the holder members 4 relative to the guns.

In this example, the movement means comprise a carriage 6 on which the clamp 5 is mounted to pivot about a pin 7 of axis that is horizontal and perpendicular to the radial plane of the platform 3 containing the axis of the corresponding gun.

The carriage 6 is mounted to slide on two rails 8 that extend vertically from the platform 3, each carriage including a portion 9 constituting a driven nut that is engaged on a vertical screw 10 fastened to an outlet shaft of a drive motor 11 for driving the carriage 6 along the rails 8. The drive motor 11 is a stepper motor.

Two actuators 12 connect the clamp 5 to the carriage 6 on either side of the pin 7 in order to form means for tilting the holder means 4 about the emitter 2 of the gun so as to enable the holder means 4 to rock about the pin 7. The actuators 12 are single-acting electromagnets. Given the orientation of the pin 7, when the holder means 4 rock, they cause the container 100 to rock about a central axis of the emitter 2 of the gun in a plane perpendicular to a travel direction of the containers. In a variant, rocking may be obtained by means of a linear cam extending along one of the rails 8.

The installation preferably includes an enclosure 50 in which the platform 3 extends together with the elements that it supports. The platform 3 has a peripheral edge that projects from the generators 1 and that is provided with an element for sealing connection with the enclosure 50. In this example, the sealing element is constituted by a baffle structure co-operating with a rib of the enclosure so as to form a barrier to prevent electrons migrating under the platform 3. The emitters 2 of the guns and the screw 10 pass through the annular portion of the platform, preferably in leaktight manner. The platform 3 defines a compartment with a controlled atmosphere above the platform 3 and a services compartment under the platform 3. By way of example, the controlled atmosphere compartment contains means for diffusing air in the form of a laminar stream from the top portion of the enclosure, and means for extracting air in order to eliminate ozone and volatile components that might be produced during sterilization.

Preferably, it is ensured that the diffusion air flows at a rate that is greater than the extraction rate so as to ensure that the top compartment is at a small positive pressure.

In a preferred embodiment, the installation also includes means for processing the inside of a container, which means act once the container is directed downwards to inject an inert gas that is lighter than the surrounding air into the inside of the container before the step of electron bombardment begins.

The injected gas is preferably nitrogen. Electron bombardment of a container that contains air gives rise to ozone being produced, so injecting nitrogen into the container before beginning electron bombardment serves to limit the amount of ozone that is formed.

Although nitrogen is very volatile in air, having the container upside-down serves advantageously to keep the injected nitrogen captive inside the container. There is therefore no need to close the container or to perform electron bombardment inside a closed enclosure that is itself filled with nitrogen, thereby making the nitrogen injection step much easier.

Naturally, the invention is not limited to the embodiment described but covers any variant coming within the ambit of the invention as defined by the claims.

In particular, the containers may be turned upside-down on the platform that carries the guns. Nevertheless this increases the size of the platform.

Although the guns are described as being stationary in height and the movement means as being arranged to vary the height of the holder members relative to the guns, the guns could be movable vertically and the movement means could be arranged to vary the height of the guns relative to the holder members.

The container holder means may be of a structure that is different from that described, for example they may be in the form of a clamp gripping the neck on either side of a midplane of the container that is perpendicular to a radial plane of the platform also containing the axis of the container.

Provision may be made to rock the holder means about axes that are radial and/or axes that are perpendicular to radial planes.

The carriage may be of swan-neck shape, the nut portion being the bottom portion of the swan neck and the clamp support portion being the top portion of the swan neck. This makes it possible to limit the length of the screw 10.

The enclosure may be arranged differently. The baffle type sealing means may be replaced by any dynamic sealing means.

The installation may be a linear installation instead of a rotary installation as described above.

The system comprising the screw 10 and the nut 9 may be replaced by a linear actuator, e.g. an electric actuator.

In a variant, no nitrogen is injected into the containers. Turning the containers upside down serves to trap the ozone that is formed inside the containers until the containers are taken to a zone of the installation that is provided with ozone extractor devices that serve to control the release of ozone out from the containers. This serves to control ozone exhaust.

What is claimed is:

1. A method of sterilizing containers by means of an electron gun, the method comprising the steps of inserting the gun into an opening of the container and applying electron bombardment, wherein the method further comprises a step of orienting the container so that its opening is directed downwards prior to inserting the gun therein.

2. A method according to claim 1, including a step, once the container has its opening directed downwards, of injecting an inert gas into the container before applying electron bombardment.

3. A container sterilization installation comprising a container transport platform, the platform having electron guns, container holder members in register with the electron guns, and relative movement means for moving the guns and the containers relative to one another in order to insert free ends of the guns into the openings of the containers, wherein the guns are directed upwards and the holder means are arranged above the electron guns.

4. An installation according to claim 3, wherein the guns are stationary vertically and the movement means are arranged to vary the height of the holder means relative to the guns.

5. An installation according to claim 4, comprising an enclosure in which the platform defines a compartment with a controlled atmosphere in which the holder members and the free ends of the guns extend, the platform having the guns passing therethrough in leaktight manner.

6. An installation according to claim 3, including tilt means for tilting the holder means about the gun.

7. An installation according to claim 6, wherein the tilt means are arranged to cause a longitudinal axis of a container to rock about a central axis of the gun in a plane perpendicular to a travel direction of the containers.

8. An installation according to claim 3, wherein the movement means comprise a carriage that is mounted to slide on at least one rail extending vertically from the platform and that supports the holder means, the movement means comprising a motor for driving the carriage along the rail.

9. An installation according to claim 8, wherein the holder means are mounted to rock about an axis that is substantially horizontal.
